(19) United States
(12) Patent Application Publication
(10) Pub. No.: US 2012/0307961 A1
(43) Pub. Date: Dec. 6, 2012

(54) IMAGE PROCESSING APPARATUS, X-RAY CT APPARATUS AND IMAGE PROCESSING METHOD

(75) Inventor: Yoshihiro IKEDA, Sakura-Shi (JP)

(73) Assignees: Toshiba Medical Systems Corporation, Otawara-Shi (JP); Kabushiki Kaisha Toshiba, Tokyo (JP)

(21) Appl. No.: 13/585,128

(22) Filed: Aug. 14, 2012

Related U.S. Application Data
(63) Continuation of application No. PCT/JP2011/075367, filed on Oct. 27, 2011.

(30) Foreign Application Priority Data
Oct. 27, 2010 (JP) 2010-240705

(51) Int. Cl. G06T 17/00; A61B 6/03

(52) U.S. Cl. 378/4; 345/424

(57) ABSTRACT

An image processing apparatus has a map data generating unit, a correction processing unit and a display processing unit. The map data generating unit generates 3D map data including voxel values based on blood signal values of a myocardium area of a heart included in volume data of the heart. The correction processing unit corrects a plurality of voxel values on each a plurality of straight lines radially extending from an interior side of the heart so as to be equivalent to a voxel value at an inner wall side of the myocardium area on each of the straight lines to generate 3D corrected map data on the basis of the 3D map data. The display processing unit generates image data on the basis of the 3D corrected map data to display on a display device.

### CROSS-REFERENCE TO RELATED APPLICATION

This application is a Continuation Application of No. PCT/JP2011/075367, filed on Oct. 27, 2011, and the PCT application is based upon and claims the benefit of priority from Japanese Patent Application No. 2010-240705, filed on Oct. 27, 2010, the entire contents of which are incorporated herein by reference.

### FIELD

The present embodiment as one aspect of the present invention relates to an image processing apparatus, an X-ray computed tomography (CT) apparatus and an image processing method for displaying myocardial perfusion data in three dimensions (3D) on the basis of volume data based on cardiac-gated scanning.

### BACKGROUND

An X-ray CT apparatus provides information about an object by images on the basis of the strength of X-ray passing through the object, and plays an important role in many medical activities such as diagnosing/treating a disease or planning surgery.

An X-ray CT apparatus is used for examining blood flow dynamics (perfusion) of a myocardium or an organ in brain tissues or the like. For these perfusion examinations, it has been experimented in research to generate perfusion data by performing dynamic scanning with bolus injection, which injects a contrast medium in a short period of time, and analyzing the obtained dynamic contrast-enhanced data.

X-ray CT apparatuses are disclosed which are capable of accurately obtaining a myocardial perfusion image in a shorter period of time without increasing the amount of a contrast medium injected into an object and the radiation exposure caused by X-ray.

Unfortunately, if a blood flow rate in the myocardium is not normal, a perfusion value of the myocardium often becomes abnormally low at an inner wall side of the myocardium while staying at a normal level at an outer wall side of the myocardium. According to the conventional art, even if original volume data and 3D perfusion map data of a left ventricle are fused and displayed in 3D, abnormal perfusion values at an inner wall side of the myocardium are covered up, and normal perfusion values at an outer side are observed. In that case, there is a technique to make it easier to visually study the abnormal inner wall side by modifying the transparency of the perfusion map data to control display. But in that case, depending on the line of sight of a 3D representation, tissues at a deeper portion of the left ventricle are displayed and abnormal perfusion values at the inner wall side cannot be observed.

Thus, when original volume data and perfusion map data of the left ventricle are directly fused and displayed in 3D, perfusion values at an inner wall side of myocardium indicating ischemia cannot be observed.

### BRIEF DESCRIPTION OF THE DRAWINGS

In accompanying drawings,

FIG. 1 is a hardware configuration diagram illustrating an X-ray CT apparatus of a present embodiment;

FIG. 2 is a block diagram illustrating functions of the X-ray CT apparatus of the present embodiment;

FIGS. 3A to 3D are diagrams illustrating a concept of generating cross-sectional data of short axis planes for a whole left ventricle area;

FIGS. 4A to 4C are diagrams illustrating a concept of generating corrected perfusion cross-sectional data on the basis of perfusion cross-sectional data;

FIG. 5 is a diagram illustrating an example of corrected perfusion volume data;

FIG. 6 is a diagram illustrating an example of 3D image data displayed; and

FIG. 7 is a flow chart illustrating behavior of the X-ray CT apparatus of the present embodiment.

### DETAILED DESCRIPTION

An image processing apparatus, an X-ray CT apparatus and an image processing method of the present embodiment will now be described with reference to accompanying drawings.

To solve the above-described problems, the present embodiments provide the image processing apparatus includes: a map data generating unit configured to generate 3D map data including voxel values based on blood signal values of a myocardium area of a heart included in volume data of the heart; a correction processing unit configured to correct a plurality of voxel values on each a plurality of straight lines radially extending from an interior side of the heart so as to be equivalent to a voxel value at an inner wall side of the myocardium area on each of the straight lines to generate 3D corrected map data on the basis of the 3D map data; and a display processing unit configured to generate image data on the basis of the 3D corrected map data to display on a display device.

To solve the above-described problems, the present embodiments provide the X-ray CT apparatus includes: an X-ray generator configured to generate X-rays; an X-ray detector configured to detect the X-rays; a volume data generating unit configured to generate volume data of a heart based on a scan using the X-ray generator and the X-ray detector; a map data generating unit configured to generate 3D map data including voxel values based on blood signal values of a myocardium area of the heart included in the volume data; a correction processing unit configured to correct a plurality of voxel values on each a plurality of straight lines radially extending from an interior side of the heart so as to be equivalent to a voxel value at an inner wall side of the myocardium area on each of the straight lines to generate 3D corrected map data on the basis of the 3D map data; and a display processing unit configured to generate image data on the basis of the 3D corrected map data to display on a display device.

To solve the above-described problems, the present embodiments provide the image processing method includes: generating 3D map data including voxel values based on blood signal values of a myocardium area of a heart included in volume data of the heart stored in a storage; correcting a plurality of voxel values on each a plurality of straight lines radially extending from an interior side of the heart so as to be equivalent to a voxel value at an inner wall side of the myocardium area on each of the straight lines to generate 3D corrected map data on the basis of the 3D map data; and generating image data on the basis of the 3D corrected map data to display on a display device.

An X-ray CT apparatus of the present embodiment is available in various types, such as a rotate/rotate type, in which an X-ray tube and an X-ray detector rotate as a unit around an object, and a stationary/rotate type, in which a large number of detection elements are arrayed in a ring form and only an X-ray tube rotates around an object, and any of these types can be used to embody the present invention. An X-ray CT apparatus of the present embodiment will hereinafter be described as the rotate/rotate type, which is the current mainstream type.

Also, a mechanism of converting incoming X-ray to electric charge is mainly available in an indirect conversion type, which converts the X-ray to a light with a phosphor such as a scintillator and then converts the light to electric charge with a photoelectric conversion element such as a photodiode, and a direct conversion type, which utilizes generation of electron-hole pairs and their movement to electrodes in a semiconductor on applying X-ray, or a photoconduction phenomenon.

In addition, in recent years, a so-called multiple tube-type X-ray CT apparatus, in which a plurality of pairs of an X-ray tube and an X-ray detector are provided on a rotary ring, has become a commercial reality and peripheral technology thereof has been under development. An X-ray CT apparatus of the present embodiment is applicable to both a conventional single tube-type X-ray CT apparatus and the multiple tube-type X-ray apparatus, and will hereinafter be described as the single tube-type X-ray CT apparatus.

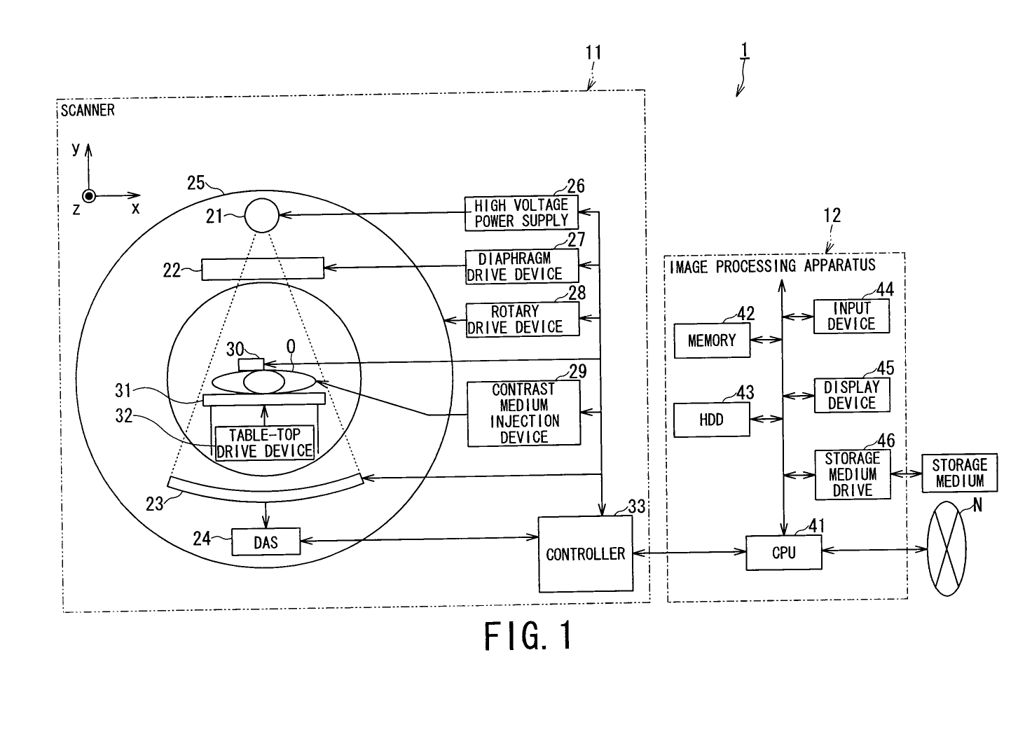

FIG. 1 is a hardware configuration diagram illustrating an X-ray CT apparatus of the present embodiment.

FIG. 1 shows an X-ray CT apparatus 1 of the present embodiment. The X-ray CT apparatus 1 largely has a scanner 11 and an image processing apparatus 12. The scanner 11 of the X-ray CT apparatus 1 is usually installed in an examination room, and configured to generate X-ray transmission data about a patient (object) O. The image processing apparatus 12 is usually installed in a control room adjacent to the examination room, and configured to generate projection data on the basis of the transmission data to generate and display a reconstructed image.

The scanner 11 of the X-ray CT apparatus 1 includes an X-ray tube (X-ray source) 21, a diaphragm 22, an X-ray detector 23, a DAS (data acquisition system) 24, a rotary member 25, a high voltage power supply 26, a diaphragm drive device 27, a rotary drive device 28, a contrast medium injection device (injector) 29, an electrocardiograph unit 30, a table-top 31, a table-top drive device 32, and a controller 33. At other times, the contrast medium injection device 29 is established outside the scanner 11.

The X-ray tube 21 generates X-ray by running an electron beam into a metal target in accordance with a tube voltage supplied from the high voltage power supply 26, and irradiates the X-ray to the X-ray detector 23. By the X-ray irradiated from the X-ray tube 21, a fan-beam X-ray or a cone-beam X-ray is formed. The X-ray tube 21 is supplied electric power required for irradiating X-ray under the control of the controller 33 via the high voltage power supply 26.

The diaphragm 22 adjusts, by way of the diaphragm drive device 27, an irradiated range of X-ray irradiated from the X-ray tube 21 in a slice direction and a direction normal to the slice direction. That is, by adjusting an aperture of the diaphragm 22 by the diaphragm drive device 27, an irradiated range of X-ray in the slice direction and the direction normal to the slice direction can be changed.

The X-ray detector 23 is a one-dimensional (1D) array-type detector having a plurality of detection elements in a channel direction while having a single detection element in a column (slice) direction. Or, the X-ray detector 23 is a two-dimensional (2D) array-type detector (also referred to as a multiple slice-type detector) having a matrix of detection elements, or a plurality of detection elements in both channel and column directions. The X-ray detector 23 detects X-ray irradiated from the X-ray tube 21 and transmitted through the patient O.

The DAS 24 amplifies signals of transmission data detected by each of the X-ray detection elements of the X-ray detector 23 and converts the signals to digital signals. Data output from the DAS 24 is supplied to the image processing apparatus 12 via the controller 33 of the scanner 11.

The rotary member 25 holds the X-ray tube 21, the diaphragm 22, the X-ray detector 23 and the DAS 24 as a unit. The rotary member 25 is configured to be capable of rotating the X-ray tube 21, the diaphragm 22, the X-ray detector 23 and the DAS 24 around the patient O as a unit, with the X-ray tube 21 and the X-ray detector 23 facing each other. In the following description, a direction parallel to the rotation center axis of the rotary member 25 will be defined as a z-axis direction, and a plane perpendicular to the z-axis direction will be defined with x-axis and y-axis directions.

The high voltage power supply 26 supplies electric power required for X-ray tube 21 to irradiate X-ray, under the control of the controller 33.

The diaphragm drive device 27 has a mechanism of adjusting an irradiated range of X-ray on the diaphragm 22 in the slice direction and the direction normal to the slice direction under the control of the controller 33.

The rotary drive device 28 has a mechanism of rotating the rotary member 25 under the control of the controller 33 so that the rotary member 25 rotates around a hollow portion while maintaining its positional relationship to the hollow portion.

The contrast medium injection device 29 continuously injects a contrast medium into the patient O under the control of the controller 33. The contrast medium injection device 29 is capable of controlling the amount and concentration of a contrast medium injected into the patient O on the basis of the behavior of the contrast medium in the patient O.

In the patient O, an aorta branches into coronaries, and coronaries further branch into capillaries. Capillaries are led into a myocardium, and the myocardium consists of capillaries and myocardial cells. Myocardial cells include an area called interstice, and blood can move between the interstice and capillaries. Thus, when a contrast medium is injected into the patient O, the contrast medium is led, along with blood, from an aorta to coronaries, and then from the coronaries to capillaries. When the contrast medium flows through the capillaries and reaches myocardial cells along with blood, some of the contrast medium flows from the capillaries into an interstice in the myocardial cells. Also, some of the blood flowed into the interstice in the myocardial cells flows out of the myocardial cells and moves back into the capillaries.

The electrocardiograph unit 30 includes not-shown electrocardiographic electrodes, an amplifier, and an A/D (analog to digital) conversion circuit. The electrocardiograph unit 30 amplifies electrocardiographic waveform data, which is electric signals sensed by the electrocardiographic electrodes, by the amplifier and eliminates noise from the amplified signals to convert to digital signals. The electrocardiograph unit 30 is worn by the patient O.

The table-top 31 is capable of being loaded with the patient O.

The table-top drive device 32 has a mechanism of moving the table-top 31 up and down along the y-axis as well as forward and backward along with the z-axis under the control of the controller 33. The rotary member 25 includes an opening portion at the center thereof, and the patient O loaded on the table-top 31 is inserted into the opening portion.

The controller 33 includes a CPU (central processing unit) and a memory. The controller 33 controls the X-ray detector 23, the DAS 24, the high voltage power supply 26, the diaphragm drive device 27, the rotary drive device 28, the contrast medium injection device 29, the electrocardiograph unit 30, the table-top drive device 32, etc. to enable scanning.

The image processing apparatus 12 of the X-ray CT apparatus 1 is configured on the basis of a computer and capable of mutually communicating with a network N such as a backbone LAN (local area network) of a hospital. The image processing apparatus 12 largely has basic hardware such as a CPU 41, a memory 42, an HDD (hard disc drive) 43, an input device 44, and a display device 45. The CPU 41 is mutually connected with each of the hardware components constituting the image processing apparatus 12 via a bus, which is a common signal transmission path. The image processing apparatus 12 may include a storage medium drive 46.

The CPU 41 is a control device having a configuration of LSI in which electronic circuits including semiconductors are enclosed in a package having a plurality of terminals. When an instruction is input by an operator such as a doctor by operating the input device 44, for example, the CPU 41 runs a program stored in the memory 42. Or, the CPU 41 loads to the memory 42 a program stored in the HDD 43, a program transmitted from the network N and installed in the HDD 43, or a program read from a recording medium loaded into the storage medium drive 46 and installed in the HDD 43, and runs the program.

The memory 42 includes a ROM (read only memory), a RAM (random access memory), and the like. This internal storage device is used for storing an IPL (initial program loader), a BIOS (basic input/output system) and data, used as a work memory of the CPU 41, or used for temporarily storing data.

The HDD 43 is a storage device having a configuration in which a metal disk coated or vapor-deposited with magnetic material is undetachably incorporated. The HDD 43 is a storage device for storing a program (not only an application program but also an OS (operating system) and the like) installed in the image processing apparatus 12 as well as projection data and image data. An OS may provide GUI (graphical user interface) which intensively uses graphic for displaying information to an operator and enables basic operations to be done with the input device 44.

The input device 44 is a pointing device operable by an operator, and sends to the CPU 41 an input signal in accordance with operation.

The display device 45 includes a not-shown image fusion circuit, a VRAM (video random access memory), a display, and the like. The image fusion circuit generates fusion data in which image data is fused with character data having various parameters or the like. The VRAM deploys the fusion data as display image data to be displayed on the display. The display may be a liquid crystal display, a CRT (cathode ray tube) display or the like and sequentially displays the display image data as a display image.

The storage medium drive 46 is capable of accepting and releasing a recording medium, and reads data (including a program) stored in the recording medium to output to the bus as well as writes data supplied via the bus to the recording medium. Such recording medium may be used for providing what is called package software.

The image processing apparatus 12 performs logarithmic conversion processing and correction processing (preprocessing) such as sensitivity correction of raw data input from the DAS 24 of the scanner 11 to generate projection data, and stores the projection data in a storage device such as the HDD 43 while correlating the projection data with phases based on electrocardiographic waveform data.

The image processing apparatus 12 also performs scattered radiation removal processing of the preprocessed projection data. The image processing apparatus 12 removes scattered radiation on the basis of the values of the projection data within an X-ray exposure range. Specifically, the image processing apparatus 12 performs scattered radiation correction of the projection data by subtracting scattered radiation, which is estimated from the size of the values of the projection data or projection data adjacent to that projection data, from the projection data.

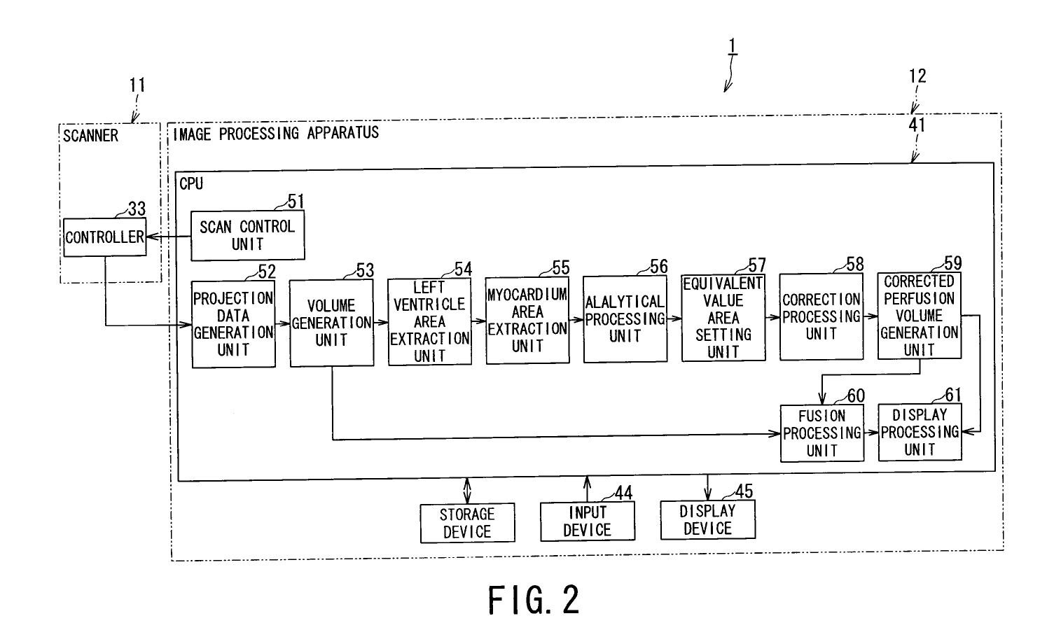

FIG. 2 is a block diagram illustrating functions of the X-ray CT apparatus 1 of the present embodiment.

By running a program with the CPU 41 shown in FIG. 1, the X-ray CT apparatus 1 (the image processing apparatus 12) performs a function of a scan control unit 51, a projection data generation unit 52, a volume generation unit 53, a left ventricle area extraction unit 54, a myocardium area extraction unit 55, an analytical processing unit (map data generation unit) 56, an equivalent value area setting unit 57, a correction processing unit 58, a corrected perfusion volume generation unit 59, a fusion processing unit 60, and a display processing unit 61. Even though each of the components 51 to 61 constituting the X-ray CT apparatus 1 functions by running a program with the CPU 41, they are not restricted to such case. All or some of the components 51 to 61 may be provided as hardware in the X-ray CT apparatus 1.

The scan control unit 51 has a function of controlling the controller 33 of the scanner 11 to continuously inject a contrast medium into the patient O while performing cardiac-gated scanning of a heart of the patient O to collect raw data with respect to each view. In other words, the scan control unit 51 controls the controller 33 to obtain electrocardiographic waveform data via the electrocardiograph unit 30 worn by the patient O, and gives a control signal based on the electrocardiographic waveform data to the high voltage power supply 26. Thus, a tube current and a tube voltage are supplied from the high voltage power supply 26 to the X-ray tube 21 in synchronization with the electrocardiographic waveform data, allowing X-ray to be irradiated to the patient O.

The projection data generation unit 52 has a function of performing logarithmic conversion processing and correction processing such as sensitivity correction of raw data input from the DAS 24 of the scanner 11 to generate projection data, and storing the projection data in a storage device such as the HDD 43. The projection data generation unit 52 may also perform scattered radiation removal processing of the projection data. The scattered radiation removal processing is a process of removing scattered radiation on the basis of the values of the projection data within an X-ray exposure range. Specifically, it corrects scattered radiation of the projection data by subtracting scattered radiation, which is estimated from the size of the values of the projection data or projection data adjacent to the projection data, from the projection data.

The volume generation unit 53 has a function of generating cross-sectional data of a plurality of cross-sectional planes perpendicular to the z-axis on the basis of projection data input from the projection data generation unit 52 (a storage device), and generating volume data on the basis of the cross-sectional data of the plurality of cross-sectional planes. Since a contrast medium is injected into the patient O, the volume data is contrast-enhanced data. Also, since cardiac-gated imaging is performed, volume data of myocardial imaging of myocardial parts in a same contracting or dilating phase of the myocardium can be obtained.

The left ventricle area extraction unit 54 has a function of extracting a left ventricle area of a heart as a volume data portion on the basis of the volume data generated by the volume generation unit 53. Although the present embodiment will be described by applying to a case where the left ventricle area of the heart is extracted as a volume data portion, it is not restricted to this case. For example, the present embodiment is applicable to a case where a right ventricle area of the heart is extracted as a volume data portion.

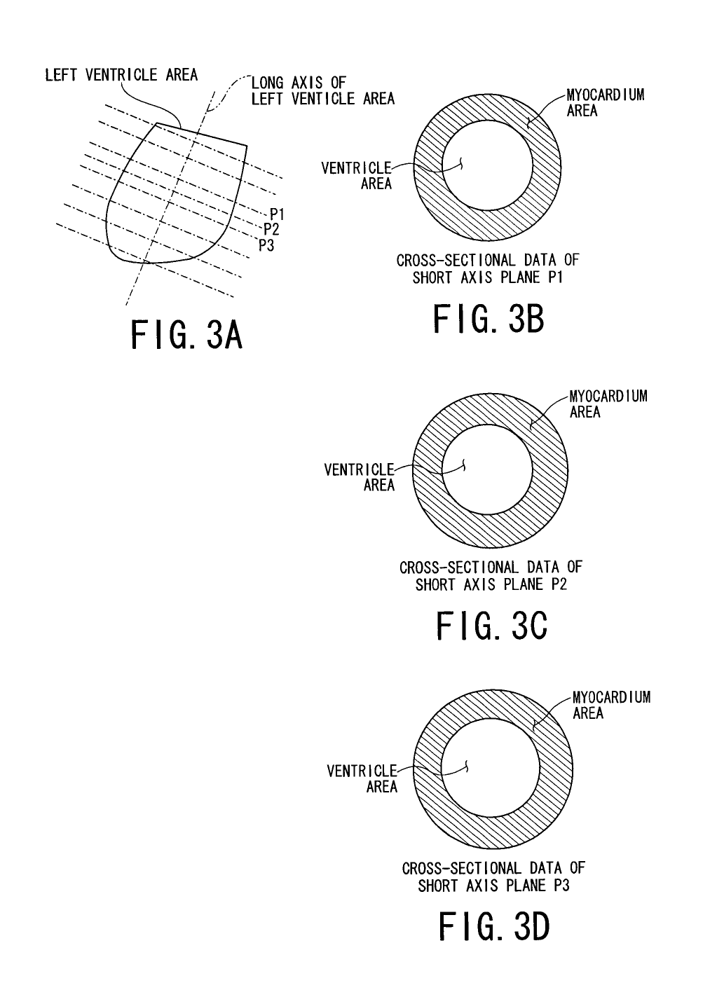

The myocardium area extraction unit 55 has a function of extracting a myocardium area on the basis of the left ventricle area of the heart extracted by the left ventricle area extraction unit 54. For example, the myocardium area extraction unit 55 extracts the myocardium area on a plurality of cross-sectional planes on the basis of the left ventricle area of the heart extracted by the left ventricle area extraction unit 54. In that case, on the basis of the left ventricle area of the heart extracted by the left ventricle area extraction unit 54, the myocardium area extraction unit 55 generates, for at least a portion of the whole left ventricle area ranging from a base portion to a middle point of an apex portion, cross-sectional data of each of a plurality of short axis planes (perpendicular-to-long axis planes), and extracts the myocardium area on the generated cross-sectional data of each of the plurality of short axis planes. For a portion ranging from the middle point of the apex portion to an apex, the myocardium area extraction unit 55 generates cross-sectional data of short axis planes as with the case of the portion ranging from the base portion to the middle point of the apex portion (shown in FIGS. 3A to 3D), or generates cross-sectional data of cross-sectional planes which, unlike the case of the portion ranging from the base portion to the middle point of the apex portion, vary in accordance with a curvature of the myocardium portion. A case will now be described in which the myocardium area extraction unit 55 extracts the myocardium area on cross-sectional data of short axis planes for the whole left ventricle area.

The myocardium area extraction unit 55 may also extract a ventricle area in addition to the myocardium area on the basis of the left ventricle area of the heart extracted by the left ventricle area extraction unit 54. Further, the myocardium area extraction unit 55 may extract the myocardium area (or the myocardium area and the ventricle area) directly from volume data generated by the volume generation unit 53 without involving the left ventricle area extraction unit 54. A case will now be described in which the myocardium area extraction unit 55 extracts only the myocardium area.

FIGS. 3A to 3D are diagrams illustrating a concept of generating cross-sectional data of short axis planes for a whole left ventricle area.

FIG. 3A shows the left ventricle area of the heart extracted by the left ventricle area extraction unit 54 and a plurality of short axis planes P1, P2 and P3 generated by the myocardium area extraction unit 55. FIG. 3B shows cross-sectional data of the short axis plane P1 of FIG. 3A. FIG. 3C shows cross-sectional data of the short axis plane P2. FIG. 3D shows cross-sectional data of the short axis plane P3. Between the cross sectional data of each of the short axis planes shown in FIGS. 3B to 3D, the myocardium area and the ventricle area are different in size. On the basis of the cross-sectional data shown in FIGS. 3B to 3D, the myocardium area extraction unit 55 extracts the myocardium area (or the myocardium area and the ventricle area) with respect to each cross-sectional data.

The analytical processing unit 56 shown in FIG. 2 has a function of generating 3D map data from voxel values based on blood signal values of the myocardium area extracted by the myocardium area extraction unit 55. The analytical processing unit 56 performs myocardial perfusion (blood flow dynamics) analytical processing on the basis of contrast medium signals as blood signal values of the myocardium area, and generates 3D perfusion map data (hereinafter referred to as “perfusion volume data”) including perfusion values as voxel values. Examples of algorithms of myocardial perfusion analytical processing include a maximum slope model and a deconvolution method.

The analytical processing unit 56 may generate 3D iodine-enhanced map data, which extracts iodine elements of a contrast medium, on the basis of blood flow signal values of the myocardium area based on volume data under different tube voltages generated in a DE (dual energy) imaging method. Also, the analytical processing unit 56 may perform myocardial perfusion analytical processing on the basis of volume data collected by a not-shown MRI device in a contrast-enhanced or nonenhanced manner to generate 3D perfusion volume data.

For example, the analytical processing unit 56 has a function of performing myocardial perfusion analytical processing on the basis of CT values (pixel values) as contrast medium signal values of the myocardium area (or the myocardium area and the ventricle area) of cross-sectional data of each cross-sectional plane extracted by the myocardium area extraction unit 55 to generate 2D perfusion map data (hereinafter referred to as “perfusion cross-sectional data”) including perfusion values as voxel values with respect to each short axis plane.

Even though in the present embodiment, the analytical processing unit 56 generates map data for the myocardium area extracted by the myocardium area extraction unit 55, this is not the only possible option. For example, the analytical processing unit 56 may generate map data for volume data generated by the volume generation unit 53 (or the left ventricle area extracted by the left ventricle area extraction unit 54), and the myocardium area extraction unit 55 may extract the myocardium area on the basis of the map data.

The equivalent value area setting unit 57 has a function of setting an equivalent value area on a same straight line among a plurality of straight lines radially extending from an interior side of the heart on the basis of perfusion cross-sectional data of a plurality of short axis plane generated by the analytical processing unit 56. The equivalent value area setting unit 57 sets a plurality of equivalent value areas for each short axis plane by dividing each perfusion cross-sectional data of a plurality of short axis planes generated by the analytical processing unit 56 into a plurality of equivalent value areas in a radial direction from a long axis center.

The correction processing unit 58 has a function of generating corrected perfusion cross-sectional data having perfusion values corrected with respect to each equivalent value area by correcting a plurality of perfusion values within an equivalent value area set by the equivalent value area setting unit 57 so as to be equivalent to a perfusion value corresponding to a low blood flow rate within the equivalent value area. The correction processing unit 58 corrects a plurality of perfusion values within an equivalent value area so as to be equivalent to a perfusion value at an inner wall side (a side close to a long axis center) of the myocardium area of the equivalent value area (that is, a perfusion value at a position closest to the inner wall of the myocardium area, an average value of a plurality of perfusion values in the myocardium area of an equivalent value area within a predetermined range from the inner wall of the myocardium area, or the like), or equivalent to a smallest perfusion value within the myocardium area of the equivalent value area.

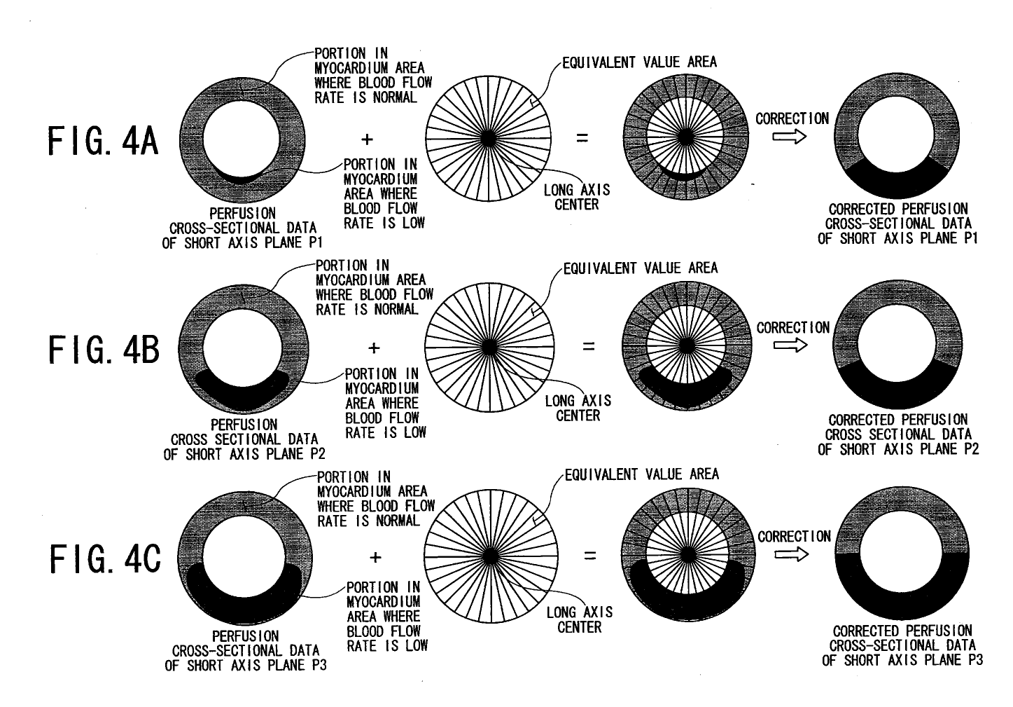

FIGS. 4A to 4C are diagrams illustrating a concept of generating corrected perfusion cross-sectional data on the basis of perfusion cross-sectional data.

Each perfusion cross-sectional data of short axis planes P1, P2 and P3 shown in FIGS. 4A to 4C is divided into a plurality of equivalent value areas, such as 32 equivalent value areas. By correcting perfusion values within each of 32 equivalent value areas of perfusion cross-sectional data of the short axis planes P1, P2 and P3 to a perfusion value at an inner wall side of the myocardium area, corrected perfusion cross-sectional data is generated as shown on the right side in FIGS. 4A to 4C.

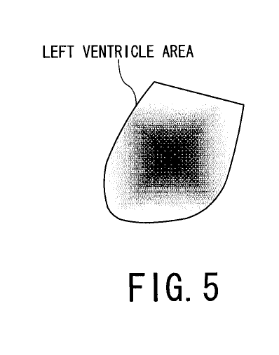

The corrected perfusion volume generation unit 59 shown in FIG. 2 has a function of generating corrected perfusion volume data on the basis of corrected perfusion cross-sectional data generated for each short axis plane by the correction processing unit 58. That is, the corrected perfusion volume generation unit 59 generates corrected perfusion volume data based on the corrected perfusion cross-sectional data shown on the right side in FIGS. 4A to 4C. On the other hand, in the conventional art, perfusion volume data is generated on the basis of the perfusion cross-sectional data on the left side in FIGS. 4A to 4C. An example of corrected perfusion volume data is shown in FIG. 5.

The fusion processing unit 60 has a function of aligning and fusing the original volume data generated by the volume generation unit 53 and the corrected perfusion volume data generated by the corrected perfusion volume generation unit 59 to generate fusion volume data.

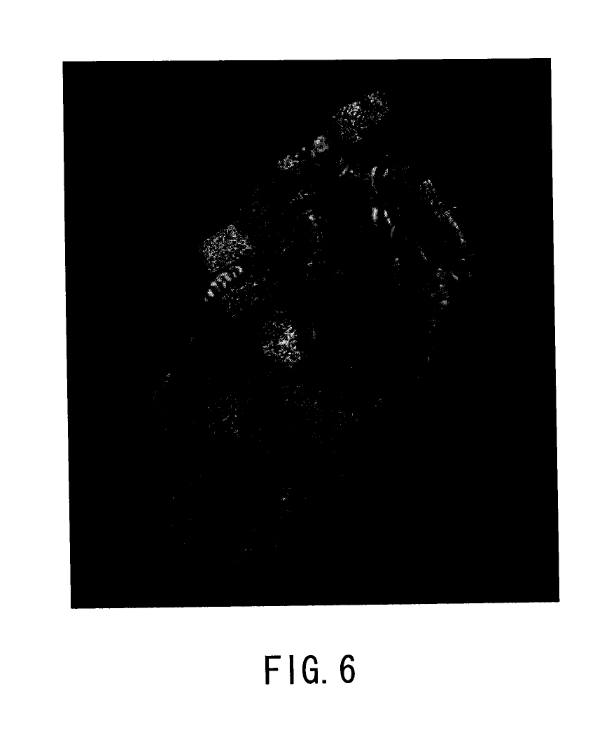

The display processing unit 61 has a function of performing volume rendering processing of the fusion volume data fused by the fusion processing unit 60 to generate 3D image data. The 3D image data generated by the display processing unit 61 is displayed on the display device 45. An example of the 3D image data displayed on the display device 45 is shown in FIG. 6. This enables observation of perfusion values representing ischemia at the inner wall side of the myocardium area which could not have been observed with the conventional 3D display.

The display processing unit 61 may perform volume rendering processing of the corrected perfusion volume data generated by the corrected perfusion volume generation unit 59 to generate 3D image data. In that case, it is preferred that the display processing unit 61 also perform volume rendering processing of original volume data generated by the volume generation unit 53 to generate 3D image data. The 3D image data based on the corrected perfusion volume data and the 3D image data based on the original volume data are then displayed in parallel or alternately on the display device 45.

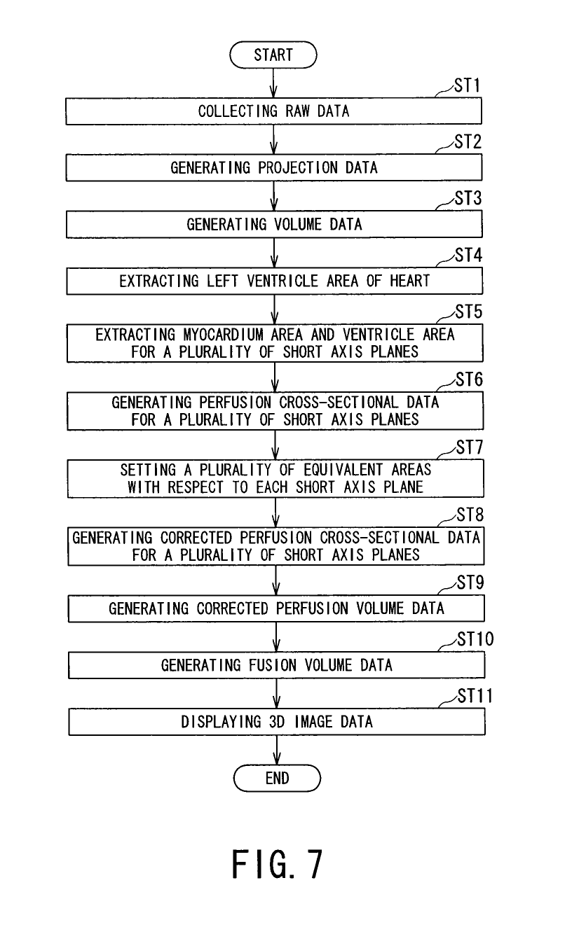

Behavior of the X-ray CT apparatus 1 of the present embodiment will now be described using a flow chart shown in FIG. 7

The X-ray CT apparatus 1 controls the controller 33 of the scanner 11 to continuously inject a contrast medium into the patient O while performing cardiac-gated scanning of a heart of the patient O to collect raw data with respect to each view (step ST1). The X-ray CT apparatus 1 performs logarithmic conversion processing and correction processing such as sensitivity correction of the raw data input from the DAS 24 of the scanner 11 to generate projection data (step ST2). On the basis of the projection data generated at step ST2, the X-ray CT apparatus 1 generates cross-sectional data of a plurality of cross-sectional planes perpendicular to the z-axis, and generates volume data on the basis of the cross-sectional data of the plurality of cross-sectional planes (step ST3).

From the volume data generated by the volume generation unit 53, the X-ray CT apparatus 1 extracts a left ventricle area of the heart as a volume data portion (step ST4). On the basis of the left ventricle area of the heart extracted at step ST4, the X-ray CT apparatus 1 generates, for at least a portion of the whole left ventricle area ranging from a base portion to a middle point of an apex portion, cross-sectional data of each of a plurality of short axis planes, and extracts a myocardium area on the generated cross-sectional data of each of the plurality of short axis planes (step ST5).

On the basis of CT values as contrast medium signal values of the myocardium area of the cross-sectional data of each cross-sectional plane extracted at step ST5, the X-ray CT apparatus 1 performs myocardial perfusion analytical processing to generate perfusion cross-sectional data including perfusion values as voxel values with respect to each short axis plane (step ST6).

On the basis of the perfusion cross-sectional data of a plurality of short axis planes generated at step ST6, the X-ray CT apparatus 1 sets an equivalent value area on a same straight line among a plurality of straight lines radially extending from an interior side of the heart (step ST7). By correcting a plurality of perfusion values within the equivalent value area set at step ST7 so as to be equivalent to a perfusion value corresponding to a low blood flow rate within the equivalent value area, the X-ray CT apparatus 1 generates corrected perfusion cross-sectional data having perfusion values corrected with respect to each equivalent value area (step ST8).

On the basis of the corrected perfusion cross-sectional data generated for each short axis plane at step ST8, the X-ray CT apparatus 1 generates corrected perfusion volume data (step ST9). The X-ray CT apparatus 1 aligns and fuses the original volume data generated at step ST3 and the corrected perfusion volume data generated at step ST9 to generate fusion volume data (step ST10). The X-ray CT apparatus 1 performs volume rendering processing of the fusion volume data fused at step ST10 to generate 3D image data and display the 3D image data on the display device 45 (step ST11).

According to the X-ray CT apparatus 1, the image processing apparatus 12 and an image processing method of the present embodiment, when fusion volume data obtained by fusing original volume data and corrected perfusion volume data is displayed in 3D, an operator can observe perfusion data representing ischemia at an inner wall side in the myocardium area without performing display control of modifying the transparency of the corrected perfusion volume data.

The X-ray CT apparatus 1 of the present embodiment is described not for restricting the present invention but for facilitating easy understanding of the present invention. Thus, each element disclosed for the X-ray CT apparatus 1 of the present embodiment is intended to include any design changes or equivalents falling within the technical scope of the present invention. For example, the image processing apparatus 12 of the X-ray CT apparatus 1 of the present embodiment may be provided in an MRI (magnetic resonance imaging) apparatus. In that case, perfusion map data is generated on the basis of original data generated by the MRI apparatus.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An image processing apparatus comprising:
a map data generating unit configured to generate 3D map data including voxel values based on blood signal values of a myocardium area of a heart included in volume data of the heart;
a correction processing unit configured to correct a plurality of voxel values on each a plurality of straight lines radially extending from an interior side of the heart so as to be equivalent to a voxel value at an inner wall side of the myocardium area on each of the straight lines to generate 3D corrected map data on the basis of the 3D map data; and
a display processing unit configured to generate image data on the basis of the 3D corrected map data to display on a display device.

2. The image processing apparatus according to claim 1, wherein
the map data generating unit performs myocardial perfusion analytical processing on the basis of contrast medium signal values as the blood signal values to generate 3D perfusion map data including perfusion values as the voxel values.

3. The image processing apparatus according to claim 2 further comprising:
a myocardium area extraction unit configured to extract a myocardium area from the volume data of the heart, wherein
the map data generating unit performs the myocardial perfusion analytical processing on the basis of contrast medium signal values of the extracted myocardium area.

4. The image processing apparatus according to claim 3, wherein
the myocardium area extraction unit generates cross-sectional data of a plurality of short axis planes for a whole area including the myocardium area on the basis of the volume data,
the map data generating unit performs the myocardial perfusion analytical processing with respect to each of the short axis planes and generates 2D perfusion map data including the perfusion values with respect to each of the short axis planes,
the correction processing unit corrects, on the basis of the 2D perfusion map data, a plurality of perfusion values on each of a plurality of straight lines radially extending on each of the short axis planes from an interior side of the heart so as to be equivalent to a perfusion value at an inner wall side of the myocardium area on each of the straight lines to generate 2D corrected perfusion map data with respect to each of the short axis planes, and
the display processing unit performs rendering processing of 3D corrected perfusion map data based on the 2D corrected perfusion map data to generate 3D image data and display the 3D image data on the display device.

5. The image processing apparatus according to claim 3, wherein
the myocardium area extraction unit generates, for at least a portion of the whole area including the myocardium area ranging from a base portion to a middle point of an apex portion, cross-sectional data of a plurality of short axis planes, and generates, for a portion ranging from the middle point of the apex portion to an apex, cross-sectional data of cross-sectional planes which vary in accordance with a curvature of a myocardium portion on the basis of the volume data,
the map data generating unit performs the myocardial perfusion analytical processing with respect to each of the cross-sectional planes and generates 2D perfusion map data including the perfusion values with respect to each of the cross-sectional planes,
the correction processing unit corrects, on the basis of the 2D perfusion map data, a plurality of perfusion values on each of a plurality of straight lines radially extending on each of the cross-sectional planes from an interior side of the heart so as to be equivalent to a perfusion value at an interior wall side of the myocardium area on each of the straight lines to generate 2D corrected perfusion map data with respect to each of the cross-sectional planes, and
the display processing unit performs rendering processing of 3D corrected perfusion map data based on the 2D corrected perfusion map data to generate 3D image data and display the 3D image data on the display device.

6. The image processing apparatus according to claim 1, wherein
the correction processing unit corrects a plurality of voxel values on each of the straight lines so as to be equivalent to a voxel value at a position closest to an inner wall on each of the straight lines.

7. The image processing apparatus according to claim 1, wherein
the correction processing unit corrects a plurality of voxel values on each of the straight lines so as to be equivalent to an average value of a plurality of voxel values on each the straight lines within a predetermined range from an inner wall of the myocardium area.

8. The image processing apparatus according to claim 1, wherein
the correction processing unit corrects a plurality of voxel values on each of the straight lines so as to be equivalent to a smallest voxel value on each of the straight lines.

9. The image processing apparatus according to claim 1 further comprising:
a fusion processing unit configured to aligns and fuses the volume data of the heart and the 3D corrected map data to generate fusion volume data, wherein
the display processing unit performs rendering processing of the fusion volume data to generate 3D image data and display the 3D image data on the display device.

10. An X-ray CT apparatus comprising:
an X-ray generator configured to generate X-rays;
an X-ray detector configured to detect the X-rays;
a volume data generating unit configured to generate volume data of a heart based on a scan using the X-ray generator and the X-ray detector;
a map data generating unit configured to generate 3D map data including voxel values based on blood signal values of a myocardium area of the heart included in the volume data;
a correction processing unit configured to correct a plurality of voxel values on each a plurality of straight lines radially extending from an interior side of the heart so as to be equivalent to a voxel value at an inner wall side of the myocardium area on each of the straight lines to generate 3D corrected map data on the basis of the 3D map data; and
a display processing unit configured to generate image data on the basis of the 3D corrected map data to display on a display device.

11. An image processing method comprising:
generating 3D map data including voxel values based on blood signal values of a myocardium area of a heart included in volume data of the heart stored in a storage;
correcting a plurality of voxel values on each a plurality of straight lines radially extending from an interior side of the heart so as to be equivalent to a voxel value at an inner wall side of the myocardium area on each of the straight lines to generate 3D corrected map data on the basis of the 3D map data; and
generating image data on the basis of the 3D corrected map data to display on a display device.
